(19) United States
(12) Patent Application Publication
(10) Pub. No.: US 2019/0254635 A1
(43) Pub. Date: Aug. 22, 2019

(54) INFORMATION-PROCESSING APPARATUS, METHOD FOR PROCESSING INFORMATION, INFORMATION-PROCESSING SYSTEM, AND NON-TRANSITORY COMPUTER-READABLE MEDIUM

(71) Applicant: CANON KABUSHIKI KAISHA, Tokyo (JP)

(72) Inventors: Shinji Ohira, Tokyo (JP); Tomokazu Tsuchimoto, Yokohama-shi (JP); Ryosuke Mizuno, Matsudo-shi (JP)

(21) Appl. No.: 16/402,108

(22) Filed: May 2, 2019

Related U.S. Application Data
(63) Continuation of application No. PCT/JP2017/042818, filed on Nov. 29, 2017.

(30) Foreign Application Priority Data
Dec. 9, 2016 (JP) 2016-239376

(51) Int. Cl. A61B 8/08; A61B 5/00; G06T 1/00; G06F 3/0482

(52) U.S. Cl. CPC A61B 8/5207; A61B 5/0095; G06F 3/0482; G06T 1/0007; A61B 5/0059

(57) ABSTRACT

An information-processing apparatus identifies plural kinds of photoacoustic images that are captured on the basis of the same photoacoustic signal and that are stored in a saving unit, and determines whether at least one of the plural kinds of photoacoustic images is to be deleted from the saving unit on the basis of information about the kind of a photoacoustic image that is included in the plural kinds of photoacoustic images that are identified.

### CROSS-REFERENCE TO RELATED APPLICATIONS

This application is a Continuation of International Patent Application No. PCT/JP2017/042818, filed Nov. 29, 2017, which claims the benefit of Japanese Patent Application No. 2016-239376, filed Dec. 9, 2016, both of which are hereby incorporated by reference herein in their entirety.

### TECHNICAL FIELD

The present disclosure relates to an information-processing apparatus, a method for processing information, and an information-processing system, and a program.

### BACKGROUND ART

In recent years, information about diagnosis and medical images that are used for the diagnosis has been computerized. PTL 1 discloses that image data is deleted after a predetermined period has elapsed since being saved in order to decrease the amount of data of the medical images that are saved in a server.

### CITATION LIST

### Patent Literature

PTL 1: Japanese Patent Laid-Open No. 2008-287653

### SUMMARY OF INVENTION

An information-processing apparatus according to an embodiment of the present invention includes an identification unit that identifies plural kinds of photoacoustic images that are captured on the basis of the same photoacoustic signal and that are stored in a saving unit, and a determination unit that determines whether at least one of the plural kinds of photoacoustic images is to be deleted from the saving unit on the basis of information about the kind of a photoacoustic image that is included in the plural kinds of photoacoustic images that are identified.

Further features of the present invention will become apparent from the following description of exemplary embodiments with reference to the attached drawings.

### BRIEF DESCRIPTION OF DRAWINGS

FIG. 1 illustrates an example of a system and a functional configuration of an information-processing apparatus according to an embodiment of the present invention.

FIG. 2 illustrates an example of an image that the information-processing apparatus according to the embodiment of the present invention causes a display unit to display.

FIG. 3 is a flowchart illustrating an example of processes that are performed by the information-processing apparatus according to the embodiment of the present invention.

FIG. 4A is a diagram for description of a photoacoustic image according to the embodiment of the present invention.

FIG. 4B is a diagram for description of the photoacoustic image according to the embodiment of the present invention.

FIG. 5A is a flowchart illustrating an example of processes that are performed by the information-processing apparatus according to the embodiment of the present invention.

FIG. 5B is a flowchart illustrating an example of processes that are performed by the information-processing apparatus according to the embodiment of the present invention.

FIG. 5C is a flowchart illustrating an example of processes that are performed by the information-processing apparatus according to the embodiment of the present invention.

FIG. 6 illustrates an example of an image that the information-processing apparatus according to the embodiment of the present invention causes the display unit to display.

FIG. 7 illustrates an example of an image that the information-processing apparatus according to the embodiment of the present invention causes the display unit to display.

FIG. 8 illustrates an example of an image that the information-processing apparatus according to the embodiment of the present invention causes the display unit to display.

FIG. 9 is a flowchart illustrating an example of processes that are performed by the information-processing apparatus according to the embodiment of the present invention.

FIG. 10 illustrates an example of a hardware configuration of the information-processing apparatus according to the embodiment of the present invention.

### DESCRIPTION OF EMBODIMENTS

Embodiments of the present invention will hereinafter be described with reference to the drawings.

### First Embodiment

In the present disclosure, an acoustic wave that is generated by expansion inside a test object when the test object is irradiated with light is referred to as a photoacoustic wave.

Attention is paid to a photoacoustic imaging to image a state of the inside of the test object in a minimally invasive manner. In the photoacoustic imaging, an organism is irradiated with pulsed light that is generated from a light source, and photoacoustic waves that are generated from a living tissue are detected after the living tissue absorbs the energy of the pulsed light that propagates and diffuses inside the organism. In the following description, an image that is imaged by using the photoacoustic waves is referred to as a photoacoustic image. In the photoacoustic imaging, a difference in light-energy absorbance between the test object such as a tumor and another tissue is used, and a transducer receives elastic waves (photoacoustic waves) that are generated when the test object absorbs the energy of the irradiated light and instantaneously expands. In the following description, a signal that is detected at this time is referred to as a photoacoustic signal. A photoacoustic imaging device can obtain distribution of optical properties in the organism, particularly, distribution of light energy absorption density by analyzing the photoacoustic signal. There are various kinds of photoacoustic images depending on the optical properties inside the test object. Example of the photoacoustic images include an absorption coefficient image that represents distribution of absorption density. An image that represents the existence or ratio of an organism molecule such as oxyhemoglobin, reduced hemoglobin, water, fat, or collagen is generated from the absorption coefficient image. For example, an image that is related to oxygen saturation, which is an indicator that represents a state of a bond between hemoglobin and oxygen, is generated on the basis of a ratio between the oxyhemoglobin and the reduced hemoglobin. The plural kinds of photoacoustic images that are generated by the photoacoustic imaging device are correlated with each other. For example, an image that represents an absorption coefficient is generated from an image that represents an initial sound pressure and an image that represents light intensity distribution.

In recent years, medical images that are used for diagnosis, including the above photoacoustic images, and various kinds of information about diagnosis have been computerized. For example, a DICOM (Digital Imaging and Communications in Medicine) standard is frequently used for information sharing between an imaging device and various devices that are connected to the imaging device. The DICOM standard defines the format of each medical image and communication protocol between the devices that use the medical image. Data that is transmitted and received in accordance with the DICOM standard is referred to as an information object (IOD or Information Object Definition). In the following description, the information object is referred to as an IOD or an object in some cases. Examples of the IOD include a medical image, patient information, inspection information, and a structured report. Various kinds of data related to inspection and treatment in which the medical image is used can be also included.

An image that is dealt with in accordance with the DICOM standard, that is, an IOD image includes metadata and image data. The metadata includes information about a patient, inspection, a series, and the image. The metadata includes an aggregate of data elements called DICOM data elements. A tag for identification of each data element is added to the corresponding DICOM data element. The image data is pixel data and has a tag for representing that this is image data. For example, a patient name in the metadata has a tag for representing that this is the name of the patient. In the case where the metadata and the image data make a DICOM data set, the IOD may also include DICOM file meta-information about the DICOM data set. The DICOM file meta-information includes, for example, information about an application that has created the IOD (DICOM file).

The photoacoustic imaging device preferably outputs an IOD photoacoustic image in accordance with the DICOM standard in order to use the photoacoustic image in various devices in a medical facility. In the photoacoustic imaging, the various kinds of photoacoustic images can be generated from the photoacoustic signal during shooting at one time as described above. However, in the case where all of the generated photoacoustic images are saved in a device, the free space of the capacity of the device may be restricted. In the case where all of plural kinds of photoacoustic images that are associated with each other are collectively deleted, however, association cannot be used to reuse the photoacoustic images.

The capacity for saving the image data can be decreased by merely deleting image data after a certain period has elapsed. However, a user cannot observe the deleted image data. An object of a first embodiment is to manage the IOD such that metadata that is related to the photoacoustic images is used to decrease the capacity for saving.

Structure of Information-Processing Apparatus

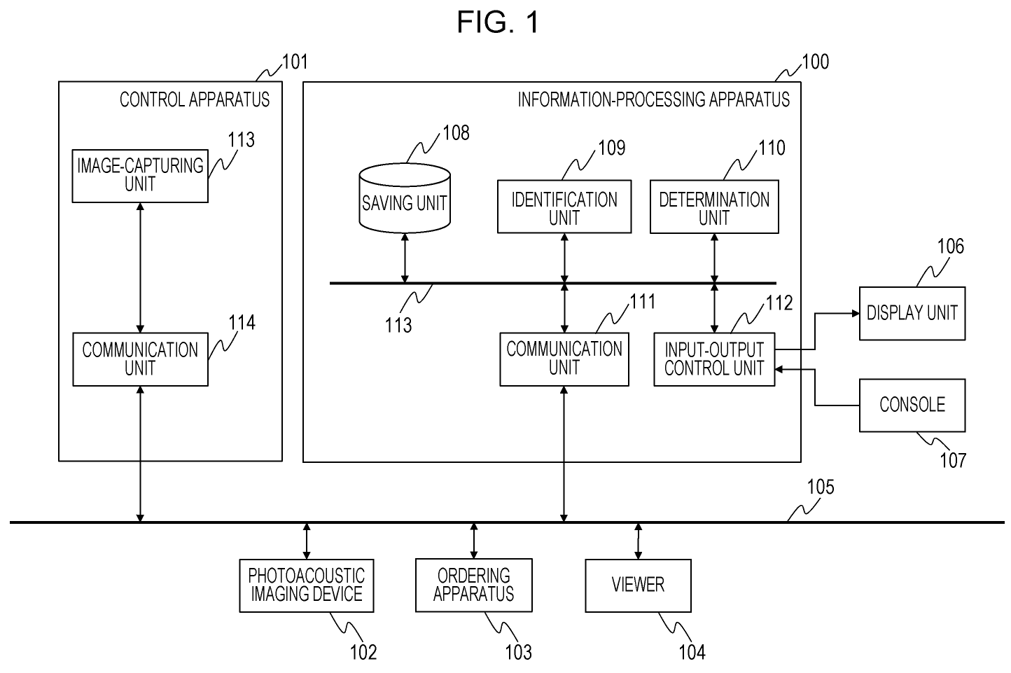

FIG. 1 illustrates an information-processing apparatus 100 according to the first embodiment and an example of the structure of a system that includes the information-processing apparatus 100. In an example illustrated in FIG. 1, the information-processing apparatus 100, a control apparatus 101, an imaging device 102, an ordering apparatus 103, and a viewer 104 are connected to each other with a network 105 interposed therebetween and included in the system. A display unit 106 and a console 107 can be connected to the information-processing apparatus 100.

An example of the imaging device 102 is a photoacoustic imaging device. The control apparatus 101 controls the imaging device 102, captures a photoacoustic image on the basis of the photoacoustic signal, and outputs an IOD photoacoustic image to the information-processing apparatus 100 or the viewer 104. An example of the information-processing apparatus 100 is a PACS (Picture Archiving and Communication System). The information-processing apparatus 100 obtains and saves the IOD that is related to the photoacoustic image. The information-processing apparatus 100 manages the saving form of plural kinds of photoacoustic images that are captured during an inspection depending on the kind thereof. Specifically, the information-processing apparatus 100 deletes the image data that can be generated on the basis of another kind of the photoacoustic image and saves only the metadata. This will now be described in detail.

The information-processing apparatus 100 includes a saving unit 108, an identification unit 109, a determination unit 110, a communication unit 111, and an input-output control unit 112.

The saving unit 108 saves the IOD and various kinds of data that are obtained from the control apparatus 101 and the imaging device 102. The saving unit 108 saves information about settings of deletion of the image data and information about a grouping process that is performed by the identification unit 109.

The identification unit 109 identifies different kinds of photoacoustic images, for example, on the basis of the metadata of the IOD that is related to the photoacoustic images that are received from the control apparatus 101. Specifically, the identification unit 109 identifies and groups the photoacoustic images that are generated on the basis of the same photoacoustic signal. For example, on the basis of information about inspection time that is written in the metadata of the IOD, the identification unit 109 determines whether the photoacoustic images are generated on the basis of the same photoacoustic signal. The identification unit 109 adds the same identifier to the photoacoustic images that are grouped and saves information about grouping such as the identifier in the saving unit 108.

The determination unit 110 determines whether the image data of the IOD that is saved in the saving unit 108 is deleted on the basis of information about the kind of the corresponding photoacoustic image and information of the group that is identified by the identification unit 109. The determination unit 110 may make the determination on the basis of information about various kinds of settings that is saved in the saving unit 108.

The communication unit 111 communicates with external devices such as the control apparatus 101 and the viewer 104 via the network 105.

The input-output control unit 112 controls the display unit 106 to cause the display unit 106 to display information. The input-output control unit 112 controls the console 107 to receive an input from the console 107.

The display unit 106 displays an image that is imaged by the photoacoustic imaging device 102 and information about inspection in response to control of the information-processing apparatus 100. The display unit 106 provides an interface for receiving a user instruction in response to control of the information-processing apparatus 100. An example of the display unit 106 is a liquid-crystal display. The console 107 transmits information about a manipulation input of a user to the information-processing apparatus 100. Examples of the console 107 include a keyboard and a mouse.

The display unit 106 and the console 107 may be integrated into a touch panel display. The display unit 106 and the console 107 may be a display unit and a console of a computer (not illustrated) that is connected to the information-processing apparatus 100 with a serial port or a network interposed therebetween provided that the information-processing apparatus 100 can input and output.

The photoacoustic imaging device 102 (also referred to below as the imaging device 102 simply) uses the photoacoustic imaging. Examples of an inner region of the targeted test object include a circulatory organ region, the breast, the groin, the abdomen, and the limbs that include the fingers and the toes. In particular, the target of each photoacoustic image to be imaged may include a blood vessel region that includes a new blood vessel and plaque on a blood vessel wall depending on characteristics that are related to light absorption inside the test object. A contrast agent may be given to a test object 1030 to image the photoacoustic image. Examples of the contrast agent include pigments such as methylene blue and indocyanine green and gold granules. An accumulation of at least one of the above substances or a substance that is chemically modified may be used as the contrast agent.

The imaging device 102 includes an irradiation unit (not illustrated) that irradiates the test object with light and a receiver (not illustrated) that receives the photoacoustic waves from the test object.

The pulse width of the light that is emitted from the irradiation unit (not illustrated) is, for example, no less than 1 ns and no more than 100 ns. The wavelength of the light that is emitted from the irradiation unit (not illustrated) is, for example, no less than 400 nm and no more than 1600 nm. In the case where a blood vessel near a surface of the test object is imaged with high resolution, the wavelength is preferably no less than 400 nm and no more than 700 nm at which the light is greatly absorbed in the blood vessel. In the case where a deep portion of the test object is imaged, the wavelength is preferably no less than 700 nm and no more than 1100 nm at which the light is unlikely to be absorbed by water and tissue such as fat. In the case where information about oxygen saturation is to be obtained, the test object is irradiated with, for example, light at a wavelength of 756 nm and light at a wavelength of 797 nm.

The receiver (not illustrated) includes at least one transducer, an example of which can detect a frequency component at, for example, 0.1 to 100 MHz. The imaging device 102 converts a time-resolved signal that is obtained by the transducer (not illustrated) into the photoacoustic signal, which is a digital signal, and transmits the converted signal to the information-processing apparatus 100.

The control apparatus 101 controls the imaging device 102. An example of the control apparatus 101 is a computer. The control apparatus 101 includes an image-capturing unit 113 and the communication unit 111.

The image-capturing unit 113 captures the photoacoustic image on the basis of the photoacoustic signal that is obtained from the imaging device 102. Specifically, the image-capturing unit 113 reconfigures distribution (referred to below as initial sound pressure distribution) of acoustic waves when light is emitted on the basis of the photoacoustic signal. The image-capturing unit 113 obtains absorption coefficient distribution of light inside the test object by dividing the reconfigured initial sound pressure distribution by light fluence distribution of the test object with respect to the light with which the test object is irradiated. For example, the light fluence distribution is obtained in advance and saved in a memory, not illustrated, which the control apparatus 101 includes. The fact that the degree of absorption of light inside the test object varies depending on the wavelength of the light with which the test object is irradiated is applied to obtain concentration distribution of a substance inside the test object from the absorption coefficient distribution relative to wavelengths. For example, the image-capturing unit 113 obtains the concentration distribution of oxyhemoglobin and deoxyhemoglobin in the substance inside the test object. The image-capturing unit 113 also obtains oxygen saturation distribution as a ratio of oxyhemoglobin concentration to deoxyhemoglobin concentration. For example, the photoacoustic image that is generated by the image-capturing unit 113 represents information about any one of or all of the initial sound pressure distribution, the light fluence distribution, the absorption coefficient distribution, the concentration distribution of the substance, and the oxygen saturation distribution, described above.

The communication unit 111 communicates with the information-processing apparatus 100 and the external devices via the network 105. For example, the communication unit 111 obtains information about the order for inspection from the ordering apparatus 103 and outputs information based on the order for the inspection to the imaging device 102. A communication unit 114 outputs, to the external device such as the information-processing apparatus 100, the data of the photoacoustic image that is captured by the image-capturing unit 113 and the IOD that includes the metadata that is related to the photoacoustic image.

The ordering apparatus 103 is a system that manages inspection information and manages the progress of the inspection by the imaging device. The inspection information includes information about an inspection ID for identification of the inspection and a shooting technique that is included in the inspection. The ordering apparatus 103 transmits information about the inspection that is carried out by the imaging device 102 to the control apparatus 101 in response to an inquiry from the control apparatus 101. The ordering apparatus 103 receives information about the progress of the inspection from the control apparatus 101.

The viewer 104 is a terminal for image diagnosis, reads the image that is stored in, for example, the information-processing apparatus 100, and displays the image for the diagnosis. A doctor observes the image that is displayed on the viewer 104 and records an image diagnosis report of information that is obtained by the observation. The image diagnosis report that is created by using the viewer 104 may be stored in the viewer 104 or may be outputted to the information-processing apparatus 100 or a report server (not illustrated) and stored therein.

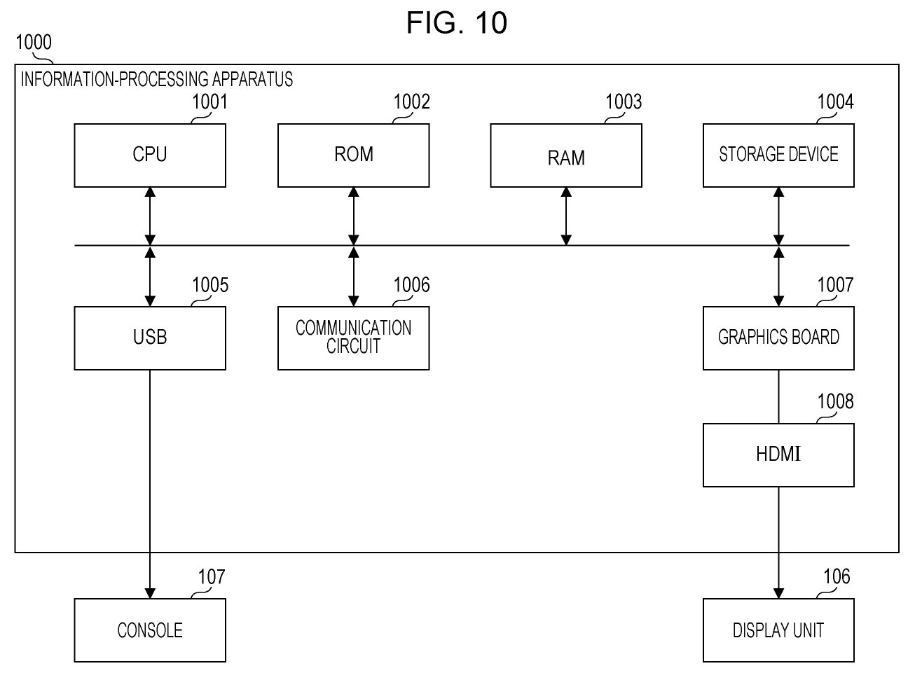

FIG. 10 illustrates an example of a hardware configuration of the information-processing apparatus 100. An example of the information-processing apparatus 100 is a server apparatus. The information-processing apparatus 100 includes a CPU 1001, a ROM 1002, a RAM 1003, a storage device 1004, a USB 1005, a communication circuit 1006, and a graphics board 1007. These are connected so as to be able to communicate by using a BUS. The BUS is used to transmit and receive data between pieces of hardware that are connected to each other and to transmit instructions from the CPU 1001 to another hardware.

The CPU (Central Processing Unit) 1001 is a control circuit that comprehensively controls the information-processing apparatus 100 and components that are connected thereto. The CPU 1001 executes programs that are stored in the ROM 1002 for the control. The CPU 1001 executes a display driver, which is software for controlling the display unit 106, for display control of the display unit 106. The CPU 1001 controls input and output for the console 107.

The ROM (Read Only Memory) 1002 stores a program in which control procedures of the CPU 1001 are written, and data. The ROM 1002 stores a boot program of the information-processing apparatus 100 and various initial data. In addition, various programs for the processes of the information-processing apparatus 100 are stored therein.

The RAM (Random Access Memory) 1003 provides a working memory area when the CPU 1001 executes an instruction program for the control. The RAM 1003 has stack and a working area. The RAM 1003 stores programs for performing the processes of the information-processing apparatus 100 and the components that are connected thereto, and various parameters that are used for an imaging process. The RAM 1003 stores a control program that is executed by the CPU 1001 and temporally stores various kinds of data for various kinds of control of the CPU 1001.

The storage device 1004 is an auxiliary storage device that saves various kinds of data such as an ultrasonic image and the photoacoustic image. Examples of the storage device 1004 include a HDD (Hard Disk Drive) and a SSD (Solid State Drive). The storage device 1004 preferably has a RAID (Redundant Arrays of Inexpensive Disks) structure.

The USB (Universal Serial Bus) 1005 is a connector that is connected to the console 107.

The communication circuit 1006 is a circuit for communication with various external devices that are connected to the components of a system 1000 and the network 105. For example, the communication circuit 1006 outputs information that is contained in a transfer packet to the external devices via the network 105 by using a communication technique such as TCP/IP. The information-processing apparatus 100 may include plural communication circuits to fit a desired communication form.

The graphics board 1007 includes a GPU (Graphics Processing Unit) and a video memory. For example, the GPU makes calculations that are related to a reconfiguration process for generating the photoacoustic image from the photoacoustic signal.

A HDMI (registered trademark) (High Definition Multimedia Interface) 1008 is a connector that is connected to the display unit 106.

The CPU 1001 and the GPU are examples of a processor. The ROM 1002, the RAM 1003, and the storage device 1004 are examples of a memory. The information-processing apparatus 100 may include plural processors. According to the first embodiment, the processor of the information-processing apparatus 100 executes the programs that are stored in the memory to perform the functions of the components of the information-processing apparatus 100.

The information-processing apparatus 100 may include a CPU, a GPU, and an ASIC (Application Specific Integrated Circuit) that exclusively perform a specific process. The information-processing apparatus 100 may include a FPGA (Field-Programmable Gate Array) in which the specific process or all of the processes are programed.

In the case where the information-processing apparatus 100 is not directly connected to the display unit 106 or the console 107, the information-processing apparatus 100 may not include the USB 1005, the graphics board 1007, or the HDMI 1008. The information-processing apparatus 100 may include a NAS (Network Attached Storage) or a SAN (Storage Area Network) that is connected to the network 105, or both instead of the storage device 1004 that the information-processing apparatus 100 includes. In any case, the information-processing apparatus 100 preferably has the RAID.

Example of Process Performed by Information-Processing Apparatus

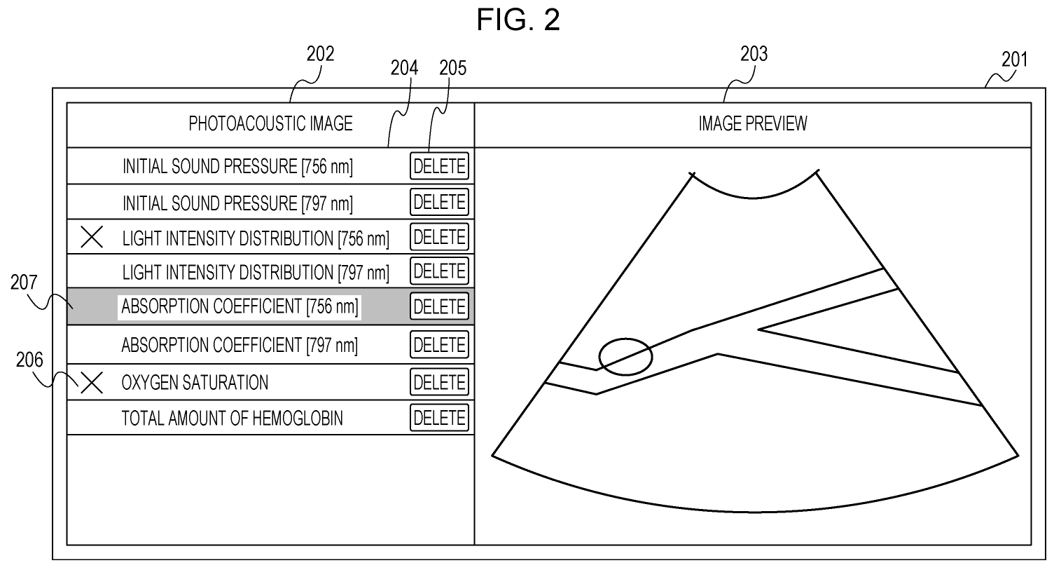

FIG. 2 illustrates an example of an image for providing a user instruction to delete the image data that is saved in the information-processing apparatus 100. An image 201 includes a list display section 202, an image display section 203, items 204, and deletion instruction sections 205.

The items 204 that are related to a series of the respective photoacoustic images that are grouped are displayed in the list display section 202. The items 204 are displayed by characters that represent the kind of the photoacoustic images. Deleted marks 206 represent that deletion is instructed by the manipulation input into the corresponding deletion instruction sections 205. The display form of an item 207 that is selected differs from that of the other items. An image that is related to the item 207 is displayed in the image display section 203. In an example illustrated in FIG. 2, the image that is related to absorption coefficient [756 nm] is displayed in the image display section 203.

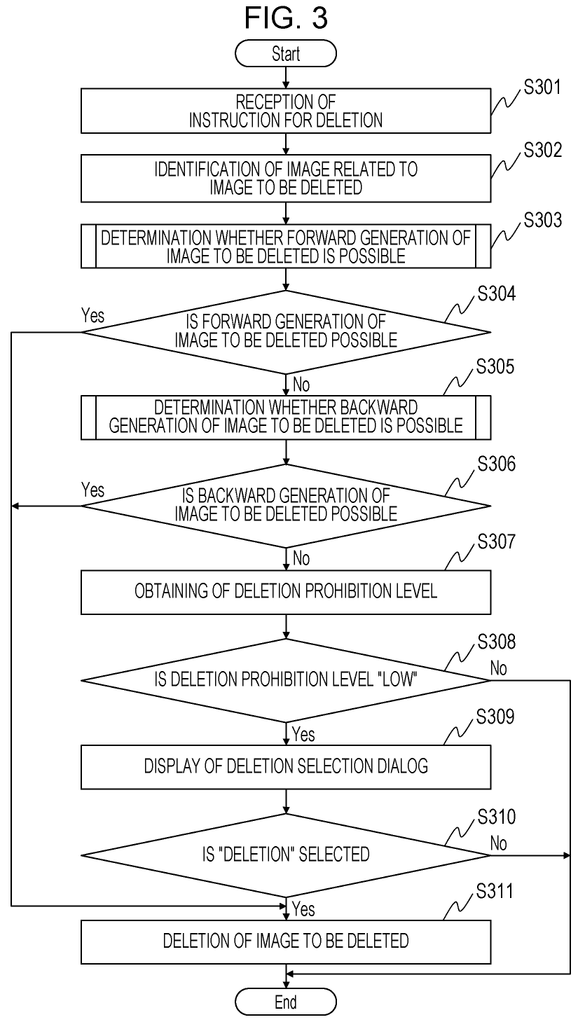

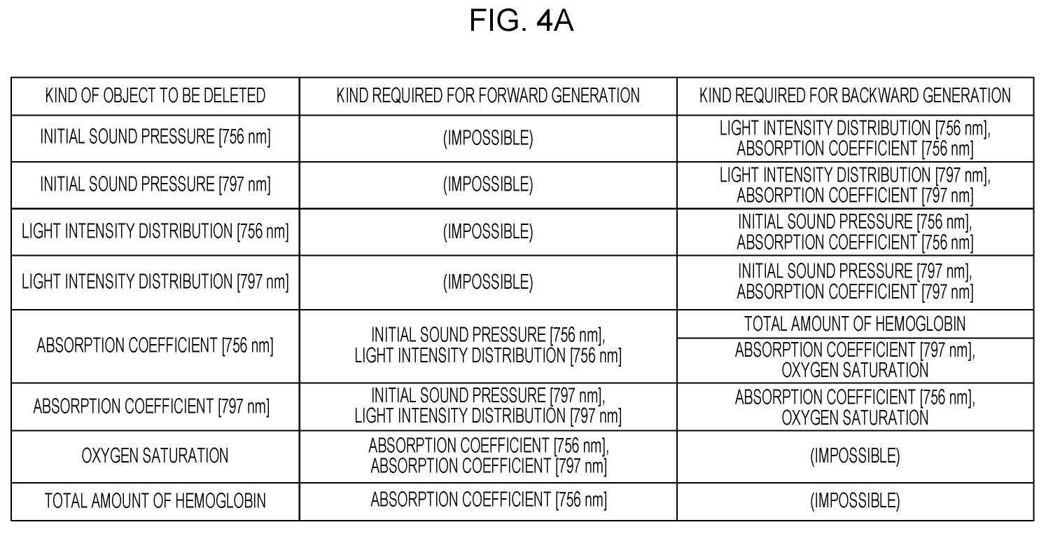

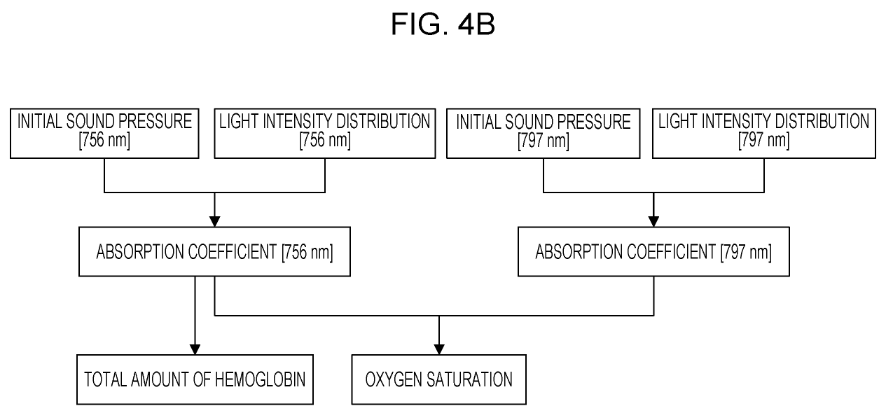

FIG. 3 is a flowchart illustrating an example of processes in the case where deletion is instructed by using a user interface in FIG. 2. The processes described below are performed mainly by the CPU 1001 or the GPU unless otherwise particularly described. The processes will be described in detail with reference to FIG. 4A to FIG. 7 appropriately.

At a step S301, the determination unit 110 receives a user instruction to delete the image data. For example, the content of the manipulation input into the corresponding deletion instruction section 205 in FIG. 2 from the console 107 is inputted into the determination unit 110 via the input-output control unit 112.

At a step S302, the identification unit 109 groups the photoacoustic images that are saved in the saving unit 108 on the basis of the metadata of the IOD that is related to the image data, deletion of which is instructed at the step S301. Specifically, the identification unit 109 identifies and groups different kinds of the photoacoustic images that are generated on the basis of the same photoacoustic signal as the image data, deletion of which is instructed. On the basis of the metadata, the identification unit 109 identifies the plural kinds of photoacoustic images that are captured on the basis of the same photoacoustic signal. For example, on the basis of information about the inspection time that is written in the metadata, the identification unit 109 identifies the photoacoustic images that are generated on the basis of the same photoacoustic signal and groups these into a group. The identification unit 109 adds the identifier to each group for management.

At a step S303, the determination unit 110 determines whether forward generation of the photoacoustic image, deletion of which is instructed at the step S301 is possible on the basis of the plural kinds of photoacoustic images that are grouped at the step S302. Specifically, the determination unit 110 determines whether part thereof is deleted from the saving unit 108 on the basis of information about the kind of each photoacoustic image of the plural kinds of photoacoustic images that are grouped. The determination whether the forward generation is possible will be described in detail later.

At a step S304, the process branches on the basis of the result of the determination at the step S303. In the case where it is determined that the forward generation is possible at the step S303, the flow proceeds to a step S311. In the case where it is determined that the forward generation is impossible, the flow proceeds to a step S305.

At the step S305, the determination unit 110 determines whether backward generation of the photoacoustic image, deletion of which is instructed at the step S301 is possible on the basis of the plural kinds of photoacoustic images that are grouped at the step S302. Specifically, the determination unit 110 determines whether part thereof is deleted from the saving unit 108 on the basis of the information of the kind of each photoacoustic image of the plural kinds of photoacoustic images that are grouped. The determination whether the backward generation is possible will be described in detail later.

At a step S306, the process branches on the basis of the result of the determination at the step S305. In the case where it is determined that the backward generation is possible at the step S305, the flow proceeds to the step S311. In the case where the backward generation is impossible, the flow proceeds to a step S307.

The determination whether the forward generation is possible and the determination whether the backward generation is possible will now be described in detail.

FIG. 4A and FIG. 4B illustrate diagrams for describing methods for generating the respective kinds of the photoacoustic images. The following description includes (1) the initial sound pressure, the light intensity distribution, the absorption coefficient that are related to the photoacoustic signal that is obtained by irradiating the test object with light at a wavelength of 756 nm, (2) the initial sound pressure, the light intensity distribution, the absorption coefficient that are related to the photoacoustic signal that is obtained by irradiating the test object with light at a wavelength of 797 nm, and (3) 8 kinds of the photoacoustic images that are related to the oxygen saturation and the total amount of hemoglobin.

FIG. 4A illustrates a table in which calculations for generating the respective kinds of the photoacoustic images and the kind of each of the photoacoustic images that are used are illustrated. The absorption coefficient is calculated on the basis of the initial sound pressure and the light intensity distribution. The oxygen saturation and the total amount of hemoglobin are calculated on the basis of the absorption coefficient. In the following description, generation of another kind of the photoacoustic image on the basis of data that is related to the initial sound pressure and the light intensity distribution is referred to as the forward generation, and generation of another kind of the photoacoustic image on the basis of data that is related to the oxygen saturation and the total amount of hemoglobin is referred to as the backward generation. In an example illustrated in FIG. 4A, a description of a specific arithmetic expression is omitted, and only the kind of data that is required for the calculation is illustrated. For example, the photoacoustic image the absorption coefficient of which is related to the photoacoustic signal that is obtained by irradiating the test object with light at a wavelength of 797 nm is generated by the forward generation from data that is related to the initial sound pressure and the light intensity distribution at a wavelength of 797 nm and is generated by the backward generation from data that is related to the oxygen saturation and the absorption coefficient (that is, the total amount of hemoglobin) at a wavelength of 756 nm.

FIG. 4B is a block diagram illustrating calculations for the forward generation of the respective kinds of the photoacoustic images. It is illustrated that the use of the photoacoustic images the kind of which is denoted by the start point of each of arrows enables the photoacoustic image the kind of which is denoted by the end point of the arrow to be generated. In the following description, the direction from the end point of each arrow toward the start point of the arrow in the block diagram in FIG. 4B is an upstream direction, and the direction from the start point of each arrow toward the end point of the arrow is referred to as a downstream direction.

When the determination unit 110 determines whether the forward generation of the photoacoustic image (referred to below as a target image) the kind of which is to be deleted, the determination unit 110 determines whether all of the photoacoustic images (referred to below as upstream images) the kind of which is ought to be adjacent to the target image in the upstream direction belong to the same group that is generated by the determination unit 110. In the example illustrated in the table in FIG. 4A, regarding a certain kind of the photoacoustic image, the upstream images correspond to the photoacoustic images the kind of which is illustrated in the column of “kind required for forward generation”. In the case where all of the upstream images belong to the same group, the determination unit 110 determines that the forward generation of the target image is possible. In the case where at least one of the upstream images does not belong to the same group, the determination unit 110 further determines whether the upstream images can be generated. That is, the determination unit 110 determines whether the photoacoustic images the kind of which is required for generating the upstream images belong to the same group on the basis of the photoacoustic images that belong to the same group. In the case where the photoacoustic images the kind of which is required for the forward generation of the upstream images and differs from the kind of the target image, or the photoacoustic images the kind of which is required for the backward generation of the upstream images and differs from the kind of the target image belong to the same group, the determination unit 110 determines that the upstream images can be generated. In the case where the upstream images can be generated, the determination unit 110 determines that the forward generation of the target image is possible. In the case where the upstream images cannot be generated on the basis of the photoacoustic images that belong to the same group, the determination unit 110 determines that the forward generation of the target image is impossible.

When the determination unit 110 determines whether the backward generation of the target image is possible, the determination unit 110 determines whether all of the photoacoustic images (referred to below as downstream images) the kind of which is adjacent to the target image in the downstream direction belong to the same group. In the example illustrated in the table in FIG. 4A, regarding a certain kind of the photoacoustic image, the downstream images correspond to the photoacoustic images the kind of which is illustrated in the column of “kind required for backward generation”. In the case where all of the downstream images belong to the same group, the determination unit 110 determines that the backward generation of the target image is possible. In the case where at least one of the downstream images does not belong to the same group, the determination unit 110 further determines whether the downstream images can be generated. That is, the determination unit 110 determines whether the photoacoustic images the kind of which is required for generating the downstream images belong to the same group on the basis of the photoacoustic images that belong to the same group. In the case where the photoacoustic images the kind of which is required for the backward generation of the downstream images and differs from the kind of the target image, or the photoacoustic images the kind of which is required for the forward generation of the downstream images and differs from the kind of the target image belong to the same group, the determination unit 110 determines that the downstream images can be generated. In the case where the downstream images can be generated, the determination unit 110 determines that the backward generation of the target image is possible. In the case where the downstream images cannot be generated on the basis of the photoacoustic images that belong to the same group, the determination unit 110 determines that the backward generation of the target image is impossible.

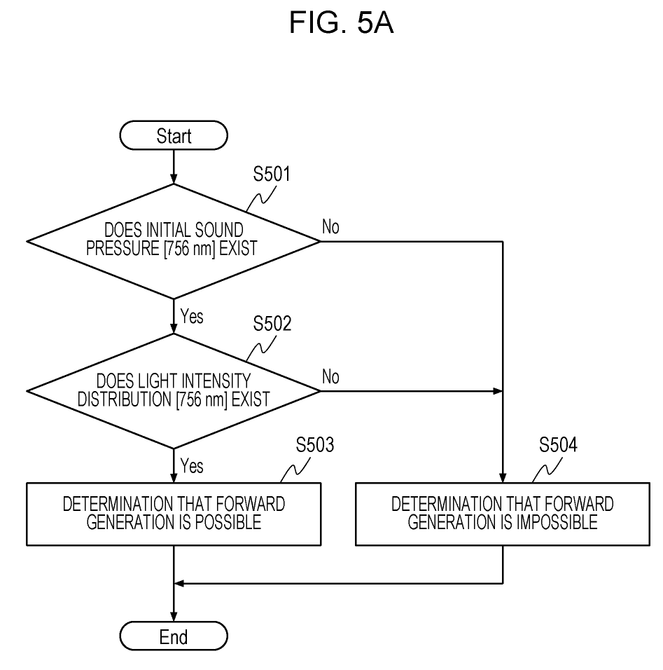

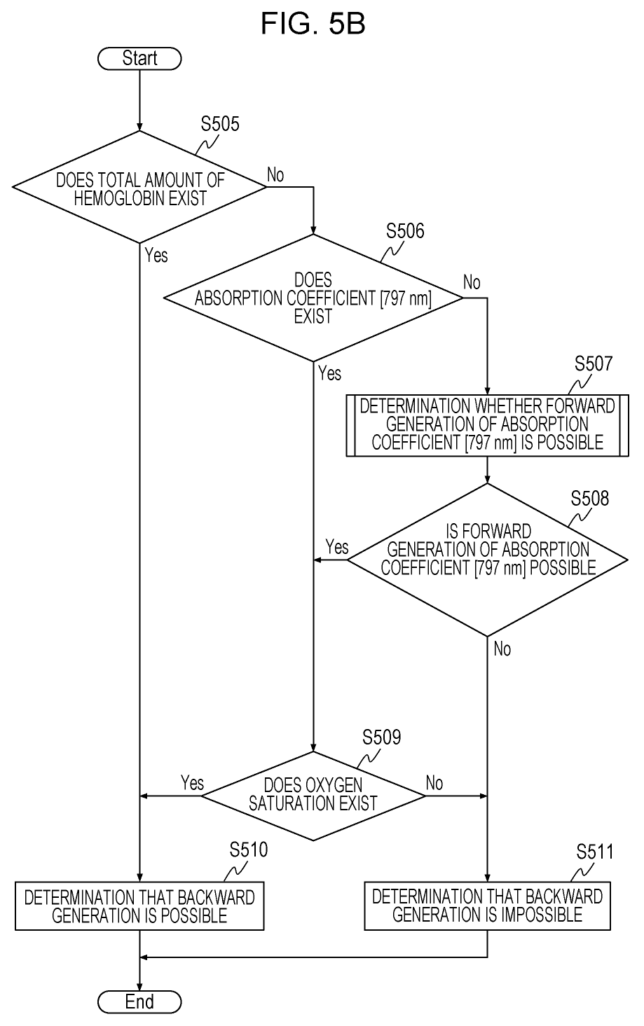

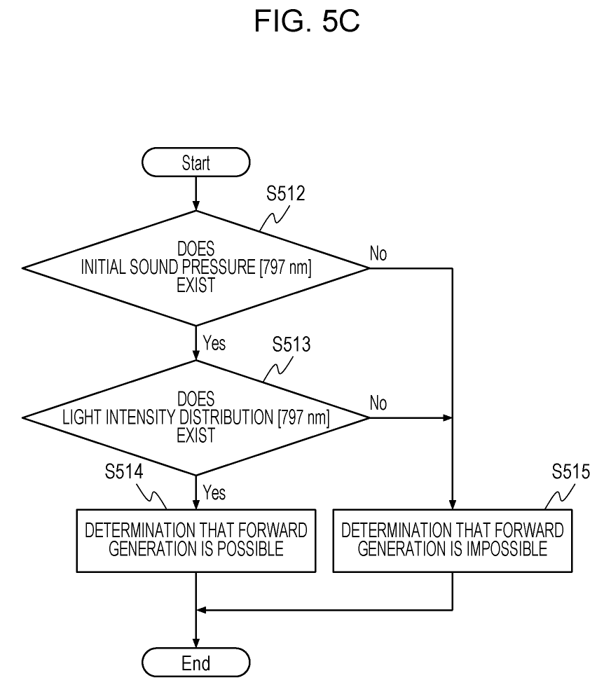

FIG. 5A, FIG. 5B, and FIG. 5C illustrate examples of processes that are performed by the determination unit 110 to determine whether the forward generation is possible and whether the backward generation is possible in the case where the target image is related to the absorption coefficient of an image that is generated on the basis of the photoacoustic signal accompanied by radiation of light at a wavelength of 756 nm. The processes described below are performed mainly by the CPU 1001 or the GPU unless otherwise particularly described. In the examples described below, the determination unit 110 makes determination about the upstream images and the downstream images of the target image as illustrated in FIG. 4A and FIG. 4B.

FIG. 5A is a flowchart illustrating an example of processes of determining whether the forward generation of the target image is possible. At a step S501 and a step S502, the determination unit 110 determines whether an image that is related to the initial sound pressure at a wavelength 756 nm and an image that is related to the light intensity distribution at a wavelength of 756 nm, which are the upstream images of the target image, belong the same group. In the case where both of the upstream images belong to the same group, the flow proceeds to a step S503, and the determination unit 110 determines that the forward generation of the target image is possible. In the case where at least one of the upstream images does not belong to the same group, the flow proceeds to a step S504, and the determination unit 110 determines that the forward generation of the target image is impossible.

FIG. 5B is a flowchart illustrating an example of processes of determining whether the backward generation of the target image is possible. At a step S505, the determination unit 110 determines whether an image that is related to the total amount of hemoglobin, which is the downstream image of the target image, belongs to the same group. In the case where the downstream image belongs to the same group, the flow proceeds to a step S510, the determination unit 110 determines that the backward generation of the target image is possible. In the case where the downstream image does not belong to the same group, the determination unit 110 determines whether images the kind of which is required for generating the downstream image belong to the same group. In the example illustrated in FIG. 5B, the photoacoustic images the kind of which is required for generating the total amount of hemoglobin and differs from the kind of the target image, which are the downstream images, are an image that is related to the absorption coefficient at a wavelength of 797 nm and an image that is related to the oxygen saturation. At a step S506, the determination unit 110 determines whether the image that is related to the absorption coefficient at a wavelength of 797 nm belongs to the same group. In the case where the image that is related to the absorption coefficient at a wavelength of 797 nm belongs to the same group, the flow proceeds to a step S509. In the case where the image that is related to the absorption coefficient at a wavelength of 797 nm does not belong to the same group, the determination unit 110 determines whether the image that is related to the absorption coefficient can be generated by using another kind of the photoacoustic image that belongs to the same group. At a step S507, the determination unit 110 determines whether the forward generation of the image that is related to the absorption coefficient at a wavelength of 797 nm is possible. The detail of the process at the step S507 is illustrated in FIG. 5C.

FIG. 5C is a flowchart illustrating an example of processes of determining whether the forward generation of the photoacoustic image at a wavelength of 797 nm is possible. At a step S512 and a step S513, the determination unit 110 determines whether an image that is related to the initial sound pressure at a wavelength of 797 nm and an image that is related to the light intensity distribution at a wavelength 797 nm, which are the upstream images of the photoacoustic image at a wavelength of 797 nm, belong to the same group. In the case where both of the upstream images belong to the same group, the flow proceeds to a step S514, and the determination unit 110 determines that the forward generation of the image that is related to the absorption coefficient at a wavelength of 797 nm is possible. In the case where at least one of the upstream images does not belong to the same group, the flow proceeds to a step S515, and the determination unit 110 determines that the forward generation of the image that is related to the absorption coefficient at a wavelength of 797 nm is impossible.

At a step S508 illustrated in FIG. 5B, the process branches on the basis of the result of the determination at the step S507. In the case where it is determined that the forward generation of the image that is related to the absorption coefficient at a wavelength of 797 nm is possible, the flow proceeds to the step S509. In the case where it is determined that the forward generation is impossible, the flow proceeds to a step S511. At the step S509, the determination unit 110 determines whether an image that is related to the oxygen saturation belongs to the same group. In the case where the image that is related to the oxygen saturation belongs to the same group, the flow proceeds to the step S510. In the case where the image does not belong to the same group, the flow proceeds to the step S511. In the example illustrated in FIG. 4B, the image that is related to the oxygen saturation is the most downstream image. The image that is related to the oxygen saturation cannot be generated by the backward generation. The target image is needed to generate the image that is related to the oxygen saturation by the forward generation. Accordingly, the determination unit 110 does not determine whether the image that is related to the oxygen saturation can be generated on the basis of the photoacoustic image that belongs to the same group. At the step S510, the determination unit 110 determines that the backward generation of the target image is possible. At the step S511, the determination unit 110 determines that the backward generation of the target image is impossible.

In the above examples, the information illustrated in FIG. 4A and FIG. 4B is saved in the saving unit 108, and the determination unit 110 reads the information to perform the processes in FIG. 5A, FIG. 5B, and FIG. 5C. The present invention is not limited thereto. The information illustrated in FIG. 4A and FIG. 4B may be saved in another location other than the information-processing apparatus 100, and the determination unit 110 may read the information. Alternatively, the control apparatus 101 may generate other photoacoustic images the kind of which is required for generating the photoacoustic images and metadata that includes information about a generating method and may transmit the IOD that includes the metadata to the information-processing apparatus 100. The determination unit 110 may perform the processes illustrated in FIG. 5A, FIG. 5B, and FIG. 5C on the basis of the information that is included in the metadata.

Returning now to the description of FIG. 3, in the case where it is determined that the forward generation, the backward generation, or both of the photoacoustic image (that is, the target image) the kind of which is instructed to be deleted at the step S301 are possible in the processes up to the step S306, the flow proceeds to the step S311, and the target image is deleted. In the case where it is determined that none of the forward generation and the backward generation of the target image is possible in the processes up to the step S306, the flow proceeds to the step S307.

At the step S307, the determination unit 110 reads information about a deletion prohibition level from the saving unit 108. The deletion prohibition level is set in advance by the user and represents information whether the deletion of the target image is permitted in the case where none of the forward generation and the backward generation of the target image is possible.

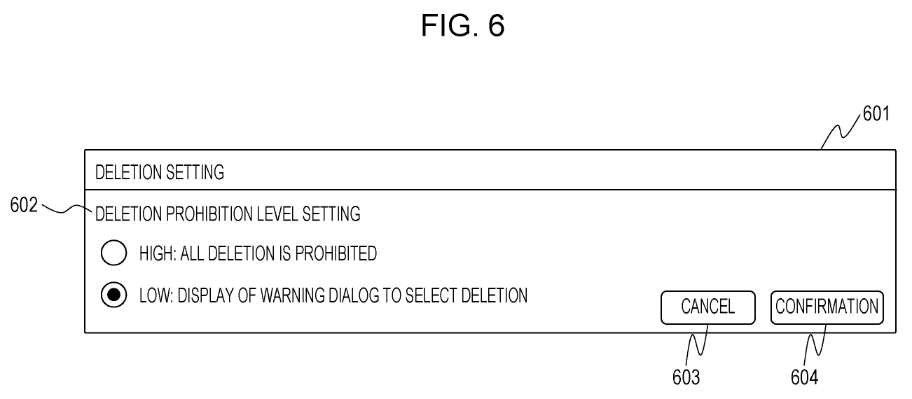

FIG. 6 illustrates an example of a setting image 601 that is displayed on the display unit 106 by the input-output control unit 112. The user sets the deletion prohibition level by using the user interface of the setting image 601. In an example illustrated in FIG. 6, the deletion prohibition level can be set at two stages of a “high” level and a “low” level. In the case where the user sets the deletion prohibition level at the “high” level, and none of the forward generation and the backward generation of the target image is possible, the determination unit 110 determines that the target image is not deleted. In the case where the user sets the deletion prohibition level at the “low” level, the determination unit 110 determines that the target image can be deleted even when none of the forward generation and the backward generation of the target image is possible.

The setting image 601 includes a level setting section 602, a cancel section 603, and a confirmation section 604. The level setting section 602 is set at either the “high” level or the “low” level as described above. The cancel section 603 is a button for canceling an edited content in the setting image 601. The confirmation section 604 is a button for confirming the edited content in the setting image 601. The confirmed content is saved in the saving unit 108.

At a step S308, the process branches depending on the setting of the deletion prohibition level that is obtained at the step S307. In the case where the level is set at the “low” level, the flow proceeds to a step S309. In the case where the level is set at the “high” level, the target image is not deleted, and the processes illustrated in FIG. 3 are finished because the target image, deletion of which is instructed at the step S301 cannot be regenerated by the forward generation or the backward generation.

At the step S309, the input-output control unit 112 causes the display unit 106 to display a dialog. The dialog is a user interface by which the user selects whether the target image is deleted.

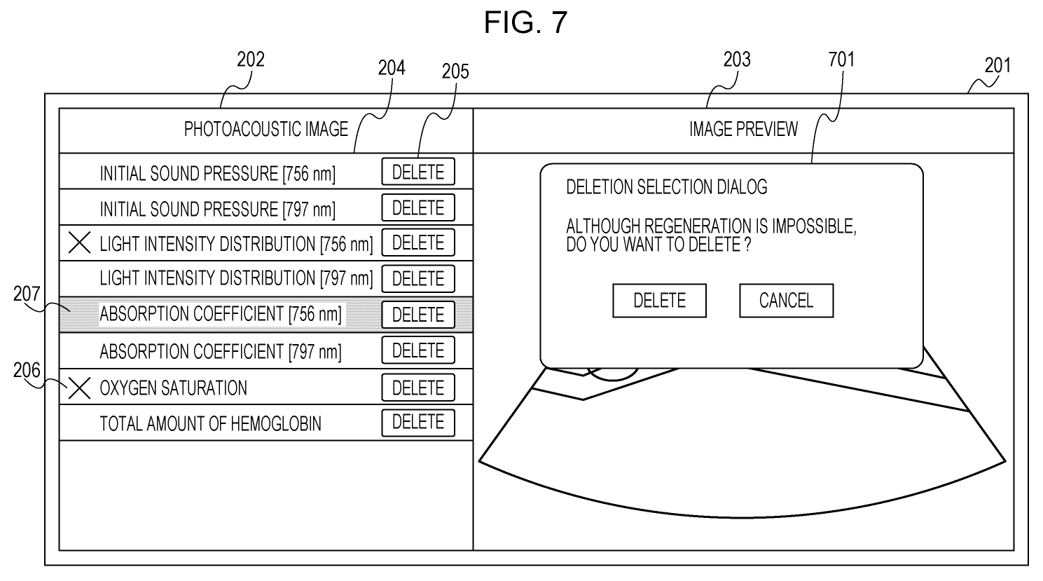

FIG. 7 illustrates an example of the dialog that is displayed on the display unit 106 at the step S309. A dialog 701 is displayed on the image 201. In an example illustrated in FIG. 7, a sentence for notifies that the target image, deletion of which is instructed by the user cannot be regenerated by using another photoacoustic image is written in the dialog 701.

At a step S310, the input-output control unit 112 obtains information about the manipulation input of the user into the dialog 701. In the case where the user decides that the target image is deleted, the flow proceeds to the step S311. In the case where the user decides that the target image is not deleted, the target image, deletion of which is instructed by the user at the step S301 is not deleted, and the processes illustrated in FIG. 3 are finished.

At the step S311, the image data of the target image, deletion of which is instructed at the step S301 is deleted from the saving unit 108. According to the first embodiment, the information-processing apparatus 100 deletes only the image data of the IOD of the target image. The determination unit 110 may add information for generating the target image into the metadata of the IOD. In the case where the user instructs deletion at the step S310, the instruction may be added into the metadata of the IOD. In another example, the information-processing apparatus 100 may delete the IOD of the target image. In this case, a method for generating the target image in the saving unit 108 may be saved in the saving unit 108.

The information-processing apparatus 100 identifies and groups the photoacoustic images that are generated on the basis of the same photoacoustic signal as described above. The information-processing apparatus 100 does not delete a group of the image data but determines whether a pieces of the image data can be regenerated from another piece of the image data to control the deletion of the image data. Since the plural kinds of the photoacoustic images are generated by different calculations, the information-processing apparatus 100 controls the deletion on the basis of calculation methods.

With the structure according to the first embodiment, it is determined that the image data that can be generated on the basis of another kind of the image data can be deleted. This enables the capacity for saving to be decreased. The control is based on the methods for generating the image data. This decreases the possibility that the image data that is required for diagnosis is mistakenly deleted.

### Modification to First Embodiment

In the example described above, whether the target image is deleted is determined on the basis of the deletion prohibition level at the step S307 to the step S310 in FIG. 3. However, the processes at the step S307 to the step S310 may not be performed. That is, the information-processing apparatus 100 may delete the target image in the case where either the forward generation or the backward generation is possible and may not delete the target image in the case where none of these is possible. The process at the step S310 may not be performed. In the case where none of the forward generation and the backward generation is possible, the information-processing apparatus 100 may not delete the target image even when the user instructs the deletion.

### Second Embodiment

In an example described according to the first embodiment, the image data is deleted by the manipulation input of the user to instruct deletion. In an example described according to a second embodiment, the image data is deleted depending on a predetermined save period.

The structure of the information-processing apparatus 100 and the structure of the system 1000 are the same as those according to the first embodiment, and the above description is referred to omit a detailed description here.

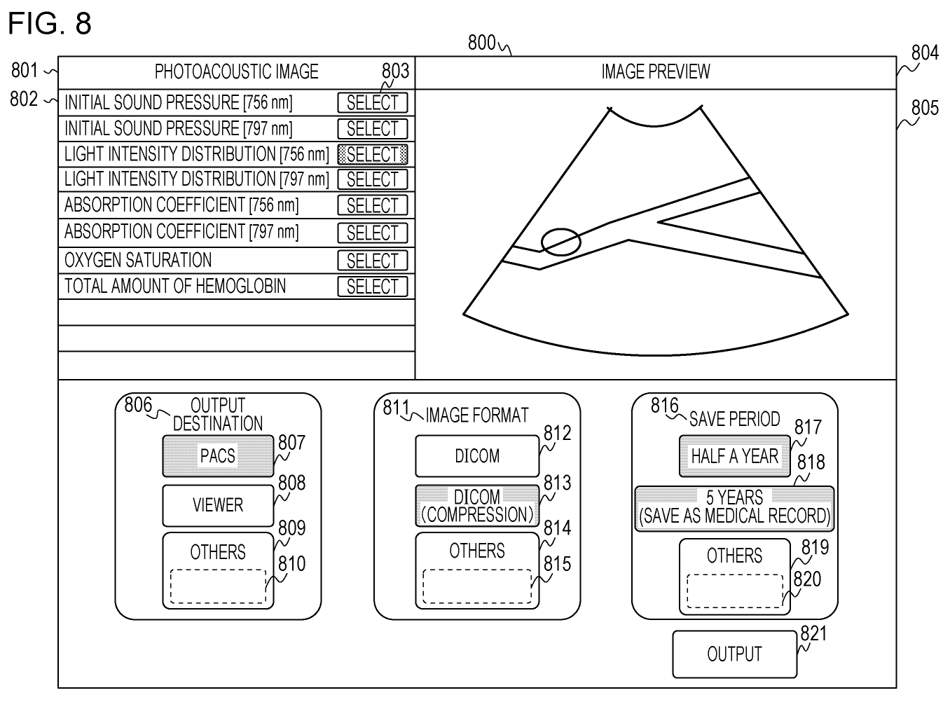

FIG. 8 illustrates an example of an image 800 that is displayed on the display unit (not illustrated) of the control apparatus 101. The image 800 is displayed on the display unit (not illustrated) when the IOD of the photoacoustic image is outputted to the external device such as the information-processing apparatus 100. The image 800 provides user interfaces by which the user selects the kind of the photoacoustic image that is outputted to the external device, the user selects the device to which the IOD is outputted, the user selects the format of the image data, and the user specifies the save period of the image data.

The kind of the photoacoustic image is displayed in a column 801. Buttons 803 for selecting whether the kind of the photoacoustic image is outputted to the external device are displayed in the rows of a data kind 802. The user can select the kind of the photoacoustic image that is outputted to the external device by the manipulation input into the corresponding button. The buttons 803 are displayed such that the buttons 803 when being selected can be distinguished from those when being not selected.

The kind that is selected by the corresponding button 803 is displayed in a region 804. The photoacoustic image the kind of which is selected by the button 803 is previewed in a region 805.

A region 806 is used to select an output destination to which the IOD that is related to the photoacoustic image the kind of which is selected by the manipulation input into the corresponding button 803 is outputted. A button 807 is used to decide that the IOD is outputted to a PACS (the information-processing apparatus 100 according to the second embodiment). A button 808 is used to decide that the IOD is outputted to the viewer 104. A button 809 is used to freely select the output destination by the user and enables the output destination to be specified by the manipulation input into a region 810.

A region 811 is used to specify the format of the image data of the photoacoustic image the kind of which is selected by the manipulation input into the buttons 803. A button 812 is used to specify a non-compression format of the image data in accordance with the DICOM standard. A button 813 is used to specify a compression format (for example, JPEG2000) of the image data in accordance with the DICOM standard. A button 814 is used to freely select the format by the user and enables the format to be specified by the manipulation input into a region 815.

A region 816 is used to specify the save period of the image data of the photoacoustic image the kind of which is selected by the manipulation input into the buttons 803. A button 817 is used to set the save period at half a year. A button 818 is used to set the save period at 5 years, that is, for a save as a medical record. A button 819 is used to freely select the save period by the user and enables the save period of the photoacoustic image the kind of which is selected to be specified by the manipulation input into a region 820. The control apparatus 101 writes information about the save period in the metadata of the IOD and outputs the information to the information-processing apparatus 100.

A button 821 is used to instruct the output of the IOD that is related to the photoacoustic image the kind of which is selected by the manipulation input into the corresponding button 803. The IOD is transmitted to the information-processing apparatus 100 in response to the manipulation input into the button 821.

The determination unit 110 of the information-processing apparatus 100 according to the second embodiment determines that the image data of the IOD is deleted when the image data of the IOD has not been read during a period that is longer than the save period that is written in the IOD.

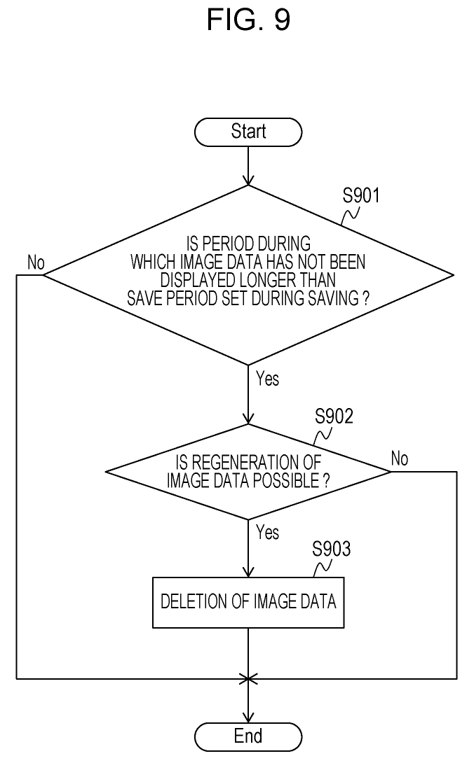

FIG. 9 is a flowchart illustrating an example of a process of deleting the image data of the IOD by the information-processing apparatus 100 that receives the IOD the save period of which is specified. The processes described below are performed mainly by the CPU 1001 or the GPU unless otherwise particularly described.

At a step S901, the determination unit 110 reads the metadata of the IOD that is saved in the saving unit 108 and obtains the information about the save period. The determination unit 110 also obtains information about history in which the image data of the IOD that is saved in the saving unit 108 has been read. Examples of the history in which the image data has been read include a history displayed on the display unit 106 that is connected to the information-processing apparatus 100 and a history outputted to the external device, such as the viewer 104, which can display the image data. According to the second embodiment, the information-processing apparatus 100 saves the information about the history in the saving unit 108. The determination unit 110 reads and obtains the information about the history from the saving unit 108.

The determination unit 110 obtains the information about the save period and information about the IOD the image data of which has not been read during a period that is longer than the save period on the basis of the history in which the image data has been read. When there is no IOD relevant to this, the processes illustrated in FIG. 9 are finished. When there is the relevant to this, the image data of the IOD is the target image to be deleted, and the flow proceeds to a step S902.

At the step S902, the determination unit 110 determines whether the target image can be regenerated. The process at the step S902 is the same as the processes at the step S301 to the step S306 illustrated in FIG. 3. When either the forward generation or the backward generation of the target image is possible, the flow proceeds to a step S903. When none of the forward generation and the backward generation of the target image is possible, the target image is not deleted, and the processes illustrated in FIG. 9 are finished.

At the step S903, the target image is deleted from the saving unit 108. Also, according to the second embodiment, the information-processing apparatus 100 may perform the process at the step S310 to cause the display unit 106 to display the dialog 701, and the user may select whether the image data is deleted. Also, according to the second embodiment, the information-processing apparatus 100 may perform the processes at the step S307 to the step S310 and may control the deletion on the basis of a predetermined deletion prohibition level.

Modification

In the examples described according to the above embodiments, the image data of the IOD is deleted. The present invention, however, is not limited thereto. For example, the IOD itself may be deleted. This enables data capacity to be decreased.

In the examples described according to the above embodiments, the image data that can be generated by using another kind of the photoacoustic image is deleted on the basis of the method for generating the image. The present invention, however, is not limited thereto. For example, only the image data that is used for diagnosis may be saved in the saving unit 108, and the other kinds of the image data may be deleted. For example, the determination unit 110 may save only the oxygen saturation and the total amount of hemoglobin that are set as the kinds that are used for diagnosis, and the other kinds of the image data may be deleted.

In the examples described according to the embodiments, the images that are related to the initial sound pressure and the light intensity distribution are the most upstream images in FIG. 4B. The present invention, however, is not limited thereto. For example, the image that is related to the absorption coefficient may be the most upstream image. In this case, the determination unit 110 may determine that the image data that is related to the oxygen saturation and the total amount of hemoglobin is deleted, for example, provided that the image data that is related to the absorption coefficient at a wavelength of 756 nm and the absorption coefficient at a wavelength of 797 nm is saved.

In the examples described according to the above embodiments, the information-processing apparatus 100 is the PACS. The present invention, however, is not limited thereto. The entire functional configuration of the information-processing apparatus 100 may be included in the control apparatus 101 that controls the imaging device 102. In this case, the control apparatus 101 may control the deletion of the image data that is saved in a PACS that is connected to the control apparatus 101. The functional configuration of the information-processing apparatus 100 may be shared by the PACS and the control apparatus 101 that controls the imaging device 102, and the above processes may be performed as a system.

The present invention can also be carried out in a manner in which the system or the apparatus is provided with a program for performing one or more functions according to the above embodiments via a network or a storage medium, and one or more processors of a computer of the system or the apparatus read and execute the program. The present invention can also be carried out by a circuit (for example, an ASIC) for performing one or more functions.

The information-processing apparatus according to each embodiment described above may be a single apparatus, or a plurality of apparatuses may be combined so as to be able to communicate with each other to perform the above processes. These are included in the embodiments of the present invention. The above processes may be performed by a common server apparatus or a server group. It is not necessary for a plurality of apparatuses that achieve the information-processing apparatus and the information-processing system to be installed in the same facility or the same country provided that the apparatuses can communicate at a predetermined communication rate.

The embodiments of the present invention include an embodiment in which the system or the apparatus is provided with a software program that performs the functions according to the above embodiments, and the computer of the system or the apparatus reads and executes codes of the provided program.

Accordingly, the program codes that are installed in the computer to perform the processes according to the embodiments by the computer are included in the embodiments of the present invention. The functions according to the above embodiments can be performed in a manner in which an OS that acts on the computer, for example, performs a part or all of actual processing on the basis of instructions that are included in the program that the computer reads.

An appropriate combination of the above embodiments is also included in the embodiments of the present invention.

The information-processing apparatus enables a part of image data to be deleted to decrease capacity for saving. Thereafter, a user can display the deleted image data.

The present invention is not limited to the above embodiments. Various modifications and alterations can be made without departing form the spirit and scope of the present invention. Accordingly, the following claims are attached to publish the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information-processing apparatus comprising:
an identification unit that identifies plural kinds of photoacoustic images that are captured on the basis of the same photoacoustic signal and that are stored in a saving unit; and
a determination unit that determines whether at least one of the plural kinds of photoacoustic images is to be deleted from the saving unit on the basis of information corresponding to the kind of a photoacoustic image in the plural kinds of photoacoustic images that are identified.

2. The information-processing apparatus according to claim 1, wherein the determination unit determines that a first photoacoustic image is to be deleted when the first photoacoustic image in the plural kinds of photoacoustic images can be generated on the basis of a second photoacoustic image that differs from the first photoacoustic image of the plural kinds of photoacoustic images.

3. The information-processing apparatus according to claim 2, wherein the determination unit determines that the first photoacoustic image is to be deleted when all of photoacoustic images that are used to generate the first photoacoustic image are included in the plural kinds of photoacoustic images that are identified.

4. The information-processing apparatus according to claim 2, wherein the determination unit determines that the first photoacoustic image is to be deleted when a third photoacoustic image that is generated by using the first photoacoustic image and all of photoacoustic images that are used to generate the third photoacoustic image and that differ from the first photoacoustic image are included in the plural kinds of photoacoustic images that are identified.

5. The information-processing apparatus according to claim 2, further comprising:
a reception unit that receives a user instruction to delete a photoacoustic image,
wherein the first photoacoustic image is the photoacoustic image, deletion of which is instructed.

6. The information-processing apparatus according to claim 2, further comprising:
a display-controlling unit that causes a display unit to display an image that is used by a user to select whether the first photoacoustic image is to be deleted when the determination unit determines that the first photoacoustic image is not to be deleted.

7. The information-processing apparatus according to claim 1, further comprising:
a reception unit that receives a user instruction to delete a photoacoustic image,
wherein the identification unit identifies a photoacoustic image that is captured on the basis of the same photoacoustic signal as the photoacoustic image, deletion of which is instructed.

8. The information-processing apparatus according to claim 1, wherein the determination unit determines whether the at least one of the plural kinds of photoacoustic images is to be deleted on the basis of a history in which the plural kinds of photoacoustic images that are stored in the saving unit are read and a predetermined save period.

9. The information-processing apparatus according to claim 8, wherein the determination unit determines that the at least one of the plural kinds of photoacoustic images is to be deleted when the at least one of the plural kinds of photoacoustic images that are stored in the saving unit has not read during a period that is longer than the predetermined save period.

10. An information-processing apparatus comprising:
a saving unit that stores plural kinds of photoacoustic images that are captured on the basis of the same photoacoustic signal; and
a delete unit that deletes at least one of the plural kinds of photoacoustic images that are stored in the saving unit,
wherein the delete unit deletes the at least one of the plural kinds of photoacoustic images such that the saving unit continuously stores a photoacoustic image that is required to generate the at least one of the plural kinds of photoacoustic images.

11. An information-processing system comprising:
an identification unit that identifies plural kinds of photoacoustic images that are captured on the basis of the same photoacoustic signal; and
a determination unit that determines whether a photoacoustic image is to be deleted on the basis of information corresponding to the plural kinds of photoacoustic images that are identified.

12. The information-processing system according to claim 11, further comprising:
a capturing unit that captures the kinds of photoacoustic images on the basis of the photoacoustic signal; and
a setting unit that sets a save period during which the kinds of photoacoustic images are saved,
wherein the determination unit determines whether the photoacoustic image is to be deleted on the basis of the set save period.

13. A method for processing information, the method comprising:
an identification step of identifying plural kinds of photoacoustic images that are captured on the basis of the same photoacoustic signal; and
a determination step of determining whether a photoacoustic image is to be deleted on the basis of information corresponding to the plural kinds of photoacoustic images that are identified.

14. A non-transitory computer-readable medium storing a program for causing a computer to execute the method for processing information according to claim 13.
